(19) United States
(12) Patent Application Publication
(10) Pub. No.: US 2007/0203114 A1
(43) Pub. Date: Aug. 30, 2007

(54) 7,8,9,10-Tetrahydro-Imidazo [2,1-A] Isochinolines

(76) Inventor: Andreas Palmer, Singen (DE)

Correspondence Address: NATH & ASSOCIATES PLLC, 112 South West Street, Alexandria, VA, 22314, US

(21) Appl. No.: 10/592,947

(22) PCT Filed: Mar. 18, 2005
(86) PCT No.: PCT/EP05/51269

§ 371 Date: Oct. 19, 2006

(30) Foreign Application Priority Data
Mar. 22, 2004 (EP) 04101167.7

(51) Int. Cl. A61K 31/5377; A61K 31/506; A61K 31/4745; C07D 471/04

(52) U.S. Cl. 514/210.21; 514/232.8; 514/256; 514/291; 544/126; 544/294; 546/80

(57) ABSTRACT

The invention provides compounds of the formula 1 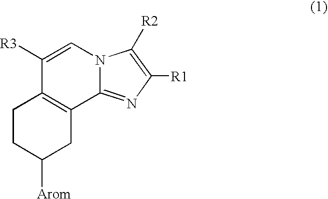 in which the substituents and symbols as provided in the description, inhibit the secretion of gastric acid.

### TECHNICAL FIELD

The invention relates to novel compounds which are used in the pharmaceutical industry as active compounds for preparing medicaments.

### PRIOR ART

U.S. Pat. No. 4,468,400 describes tricyclic imidazo[1,2-a]pyridines having different ring systems fused to the imidazopyridine skeleton, which compounds are said to be useful for treating peptide ulcer disorders.

The international patent application WO 03/014123 discloses tricyclic imidazopyridine derivatives having a very specific substitution pattern, which compounds are likewise said to be suitable for treating gastrointestinal disorders.

Kaminski et al, J. Med. Chem. 1997, 40, 427 describe the synthesis of imidazo[1,2-a]pyridines and their use as antiulcer agents.

### DESCRIPTION OF THE INVENTION

The invention provides compounds of the formula 1 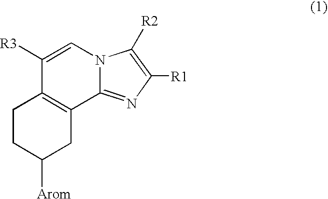
in which
R1 is hydrogen, 1-4C-alkyl, 3-7C-cycloalkyl, 1-4C-alkoxy-1-4C-alkyl or 1-4C-alkoxycarbonyl, 2-4C-alkenyl, 2-4C-alkinyl, fluoro-1-4C-alkyl or hydroxy-1-4C-alkyl
R2 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, hydroxy-3-4-C-alkenyl, hydroxy-3-4C-alkinyl, 1-4C-alkoxycarbonyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
   where
   R4 is hydrogen, 1-4C-alkyl, hydroxy-1-4C-alkyl, 1-4C-alkoxy, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxyl,
   R6 is hydrogen, 1-4C-alkyl or halogen and
   R7 is hydrogen, 1-4C-alkyl or halogen,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
      and the salts of these compounds.

1-4C-Alkyl denotes straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-4C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl denotes one of the abovementioned 1-4C-alkyl radicals which is substituted by one or more fluorine atoms. An example which may be mentioned is the trifluoromethyl radical.

Hydroxy-1-4C-alkyl denotes abovementioned 1-4C-alkyl radicals which are substituted by a hydroxyl group. Examples which may be mentioned are the hydroxymethyl, the 2-hydroxyethyl and the 3-hydroxypropyl radicals.

3-4C-Alkenyl denotes straight-chain or branched alkenyl radicals having 3 to 4 carbon atoms. Examples which may be mentioned are the 2-butenyl, 3-butenyl, 1-propenyl and the 2-propenyl (allyl) radicals.

3-4C-Alkynyl denotes straight-chain or branched alkynyl radicals having 3 to 4 carbon atoms. Examples which may be mentioned are the 2-butynyl, the 3-butynyl and, preferably, the 2-propynyl (propargyl radicals).

Hydroxy-3-4-C-alkenyl denotes abovementioned 3-4-C-alkenyl radicals which are substituted by a hydroxyl group. Examples which may be mentioned are the 1-hydroxypropenyl or the 1-hydroxy-2-butenyl radical.

Hydroxy-3-4-C-alkinyl denotes abovementioned 3-4-C-alkinyl radicals which are substituted by a hydroxyl group. Examples which may be mentioned are the 1-hydroxypropinyl or the 1-hydroxy-2-butinyl radical.

For the purpose of the invention, halogen is bromine, chlorine and fluorine.

1-4C-Alkoxy-1-4C-alkoxy denotes one of the abovementioned 1-4C-alkoxy radicals which is substituted by a further 1-4C-alkoxy radical. Examples which may be mentioned are the radicals 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl denotes one of the abovementioned 1-4C-alkoxy-1-4C-alkyl radicals which is substituted by one of the abovementioned 1-4C-alkoxy radicals. An example which may be mentioned is the radical 2-(methoxy)ethoxymethyl (CH₃—O—CH₂—CH₂—O—CH₂—).

1-7C-Alkyl denotes straight-chain or branched alkyl radicals having 1 to 7 carbon atoms. Examples which may be mentioned are the heptyl, isoheptyl-(5-methylhexyl), hexyl, isohexyl-(4-methylpentyl), neohexyl-(3,3-dimethylbutyl), pentyl, isopentyl-(3-methylbutyl), neopentyl-(2,2-dimethylpropyl), butyl, isobutyl, sec-butyl, tert-butyl, propyl, isopropyl, ethyl and methyl radicals.

1-4C-Alkylcarbonyl denotes a radical which, in addition to the carbonyl group, contains one of the abovementioned 1-4C-alkyl radicals. An example which may be mentioned is the acetyl radical.

2-4-C-Alkenylcarbonyl denotes a radical which, in addition to the carbonyl group, contains one of the abovementioned 2-4C-alkenyl radicals. An example which may be mentioned is the ethenylcarbonyl or the 2-propenylcarbonyl radical.

2-4-C-Alkinylcarbonyl denotes a radical which, in addition to the carbonyl group, contains one of the abovementioned 2-4C-alkinyl radicals. An example which may be mentioned is the ethinylcarbonyl or the 2-propinylcarbonyl radical.

Carboxy-1-4C-alkyl denotes, for example, the carboxymethyl (—CH₂COOH) or the carboxyethyl (—CH₂CH₂COOH) radical.

1-4C-Alkoxycarbonyl-1-4C-alkyl denotes one of the abovementioned 1-4C-alkyl radicals which is substituted by one of the abovementioned 1-4C-alkoxycarbonyl radicals. An example which may be mentioned is the ethoxycarbonylmethyl (CH₃CH₂OC(O)CH₂—) radical.

Aryl-1-4C-alkyl denotes an aryl-substituted 1-4C-alkyl radical. An example which may be mentioned is the benzyl radical.

Aryl-1-4C-alkoxy denotes an aryl-substituted 1-4C-alkoxy radical. An example which may be mentioned is the benzyloxy radical.

Mono- or di-1-4C-alkylamino radicals contain, in addition to the nitrogen atom, one or two of the abovementioned 1-4C-alkyl radicals. Preference is given to di-1-4C-alkylamino and in particular to dimethyl-, diethyl- or diisopropylamino.

Mono- or di-1-4C-alkylamino-1-4C-alkyl denotes one of the abovementioned 1-4C-alkyl radicals which is substituted by one of the abovementioned mono- or di-1-4C-alkylamino radicals. Preferred mono- or di-1-4C-alkylamino-1-4C-alkyl radicals are the mono- or di-1-4C-alkylaminomethyl radicals. An Example which may be mentioned is the dimethylaminomethyl (CH₃)₂N—CH₂ radical.

1-4C-Alkylcarbonylamino denotes an amino group to which a 1-4C-alkylcarbonyl radical is attached. Examples which may be mentioned are the propionylamino (C₃H₇C(O)NH—) and the acetylamino (acetamido, CH₃C(O)NH—) radicals.

1-4C-Alkoxycarbonylamino denotes an amino radical which is substituted by one of the abovementioned 1-4C-alkoxycarbonyl radicals. Examples which may be mentioned are the ethoxycarbonylamino and the methoxycarbonylamino radicals.

1-4C-Alkoxy-1-4C-alkoxycarbonyl denotes a carbonyl group to which one of the abovementioned 1-4C-alkoxy-1-4C-alkoxy radicals is attached. Examples which may be mentioned are the 2-(methoxy)-ethoxycarbonyl (CH₃—O—CH₂CH₂—O—CO—) and the 2-(ethoxy)ethoxycarbonyl (CH₃CH₂—O—CH₂CH₂—O—CO—) radicals.

1-4C-Alkoxy-1-4C-alkoxycarbonylamino denotes an amino radical which is substituted by one of the abovementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radicals.

Radicals Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyl-oxyphenyl, 3-benzyloxy-4-methoxyphenyl, 4-benzyloxy-3-methoxyphenyl, 3,5-bis-(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chloro-phenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-tri-fluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chloro-benzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxy-carbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

The compounds of the formula 1 have at least one center of chirality in the skeleton. The invention thus provides all feasible enantiomers in any mixing ratio, including the pure enantiomers, which are the preferred subject matter of the invention.

Compounds which are to be mentioned are those compounds of the formula 1, where
R1 is 1-4C-alkyl or 3-7C-cycloalkyl,
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di-1-4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are to be particularly mentioned are those compounds of the formula 1, where
R1 is 1-4C-alkyl or 3-7C-cycloalkyl,
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di-1-4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are preferred, are those compounds of the formula 1, where
R1 is 1-4C-alkyl or 3-7C-cycloalkyl
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di-1-4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl or 1-4C-alkoxy-1-4C-alkyl or 3-7C-cycloalkyl
      and
   R32 is hydrogen, 1-7C-alkyl, hydroxy-1-4C-alkyl or 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds, which are also preferred, are those compounds of the formula 1, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are also to be mentioned are those compounds of the formula 1, where
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR2R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are also to be particularly mentioned are those compounds of the formula 1, where
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also preferred, are those compounds of the formula 1, where
R1 is 1-4C-alkyl or 3-7C-cycloalkyl
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl or 1-4C-alkoxy-1-4C-alkyl or 3-7C-cycloalkyl
      and
   R32 is hydrogen, 1-7C-alkyl, hydroxy-1-4C-alkyl or 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds, which are also preferred, are those compounds of the formula 1, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also particularly preferred, are those compounds of the formula 1, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also to be emphasized, are those compounds of the formula 1, where
R1 is 1-4C-alkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds which are to be particularly emphasized are those of the formula 1 in which
R1 is 1-4C-alkyl
R2 is hydrogen, 1-4C-alkyl, halogen, hydroxy-1-4C-alkyl
R3 is hydroxy-1-4C-alkyl, 1-4C-alkoxy-1-4C-alkyl, carboxyl, 1-4C-alkoxycarbonyl or the radical —CO—NR31R32
   where
   R31 is hydrogen, 1-4C-alkyl, hydroxy-1-4C-alkyl or 1-4C-alkoxy-1-4C-alkyl
   R32 is hydrogen, 1-4C-alkyl
   or where
   R31 and R32 together and including the nitrogen atom to which they are attached form a pyrrolidino or morpholino radical
Arom is phenyl
   and the salts of these compounds.

Compounds which are also to be particularly emphasized are those of the formula 1 in which
R1 is 1-4C-alkyl
R2 is hydrogen, 1-4C-alkyl, halogen, hydroxy-1-4C-alkyl
R3 is carboxyl, 1-4C-alkoxycarbonyl or the radical —CO—NR31R32
   where
   R31 is hydrogen, 1-4C-alkyl, hydroxy-1-4C-alkyl or 1-4C-alkoxy-1-4C-alkyl
   R32 is hydrogen, 1-4C-alkyl
   or where
   R31 and R32 together and including the nitrogen atom to which they are attached form a pyrrolidino or morpholino radical
Arom is phenyl
   and the salts of these compounds.

Among the compounds of the formula 1, those compounds of the formula 1-a are preferred. 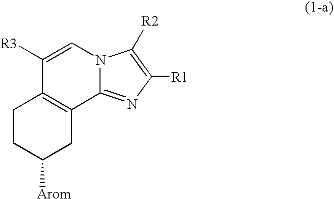
in which R1, R2, R3 and Arom have the meanings as indicated in the outset.

Compounds which are to be mentioned are those compounds of the formula 1-a, where
R1 is 1-4C-alkyl or 3-7C-cycloalkyl,
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di-1-4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are to be particularly mentioned are those compounds of the formula 1-a, where
R1 is 1-4C-alkyl or 3-7C-cycloalkyl,
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di-1-4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are preferred, are those compounds of the formula 1-a, where
R1 is 1-4C-alkyl or 3-7C-cycloalkyl
R2 is hydrogen, 1-4C-alkyl, halogen, 2-4C-alkenyl, 2-4C-alkynyl, hydroxy-1-4C-alkyl, 1-4C-alkoxycarbonyl, cyanomethyl, carboxyl, mono- or di--4C-alkylamino-1-4C-alkyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl or 1-4C-alkoxy-1-4C-alkyl or 3-7C-cycloalkyl
      and
   R32 is hydrogen, 1-7C-alkyl, hydroxy-1-4C-alkyl or 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds, which are also preferred, are those compounds of the formula 1-a, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are also to be mentioned are those compounds of the formula 1-a, where
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds which are also to be particularly mentioned are those compounds of the formula 1-a, where
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also preferred, are those compounds of the formula 1-a, where
R1 is 1-4C-alkyl or 3-7C-cycloalkyl
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical.
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl or 1-4C-alkoxy-1-4C-alkyl or 3-7C-cycloalkyl
      and
   R32 is hydrogen, 1-7C-alkyl, hydroxy-1-4C-alkyl or 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds, which are also preferred, are those compounds of the formula 1-a, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also particularly preferred, are those compounds of the formula 1-a, where
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is a R4-, R5- and R6-substituted phenyl,
   where
   R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl, trifluoromethyl,
   R5 is hydrogen, 1-4C-alkyl, halogen, or hydroxyl and
   R6 is hydrogen or 1-4C-alkyl
      and the salts of these compounds.

Compounds, which are also to be emphasized, are those compounds of the formula 1-a, where
R1 is 1-4C-alkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached are a pyrrolidino, piperidino, morpholino, aziridino or azetidino radical,
Arom is phenyl
   and the salts of these compounds.

Compounds which are to be particularly emphasized are those of the formula 1-a in which
R1 is 1-4C-alkyl
R2 is hydrogen, 1-4C-alkyl, halogen, hydroxy-1-4C-alkyl
R3 is hydroxy-1-4C-alkyl, 1-4C-alkoxy-1-4C-alkyl, carboxyl, 1-4C-alkoxycarbonyl or the radical —CO—NR31R32
   where
   R31 is hydrogen, 1-4C-alkyl, hydroxy-1-4C-alkyl or 1-4C-alkoxy-1-4C-alkyl
   R32 is hydrogen, 1-4C-alkyl
   or where
   R31 and R32 together and including the nitrogen atom to which they are attached form a pyrrolidino or morpholino radical
Arom is phenyl
   and the salts of these compounds.

Compounds which are also to be particularly emphasized are those of the formula 1-a in which
R1 is 1-4C-alkyl
R2 is hydrogen, 1-4C-alkyl, halogen, hydroxy-1-4C-alkyl
R3 is carboxyl, 1-4C-alkoxycarbonyl or the radical —CO—NR31R32
   where
   R31 is hydrogen, 1-4C-alkyl, hydroxy-1-4C-alkyl or 1-4C-alkoxy-1-4C-alkyl
   R32 is hydrogen, 1-4C-alkyl
   or where
   R31 and R32 together and including the nitrogen atom to which they are attached form a pyrrolidino or morpholino radical
Arom is phenyl
   and the salts of these compounds.

The compounds of the formula 1-a according to the invention can be obtained in a manner familiar to the person skilled in the art, for example by enantioselective synthesis, or from the corresponding racemic mixture by chromatographic separation on chiral separation columns, by derivatization with chiral auxiliaries, subsequent separation of the diastereomers and removal of the chiral auxiliary group, by salt formation with chiral acids, subsequent separation of the salts and liberation of the desired compound from the salt, or by (fractional) crystallization from a suitable solvent.

The compounds according to the invention can be synthesized for example according to the general procedure shown in Scheme 1 using 7-aryl substituted tetrahydroisoquinolines of the formula 2 as starting materials. The amino substituent can be introduced for example by nucleophilic substitution, for example by Chichibabin reaction using sodium amide. The obtained amino substituted intermediates of the formula 3 can then be further functionalized by electrophilic aromatic substitution. An example to be mentioned is the preparation of compounds of the formula 4 with R3=halogen, for example Br, which are very useful starting materials for the synthesis of compounds of the formula 1 with R3=halogen, for example Br. These derivatives can be obtained from compounds of the formula 3 by reaction with a suitable halogenation reagent, for example a bromination reagent like N-bromosuccinimide. The anellation of the imidazole ring can be accomplished by condensation of compounds of the formula 4 with α-functionalized ketones, whereby X is a suitable leaving group, like for example a halogen atom, for example bromine or chlorine. A related synthesis for compounds of the formula 1 with R3=hydrogen is described in U.S. Pat. No. 4,468,400. The synthesis is carried out in a manner known to the expert, for example as described in more detail in the examples.

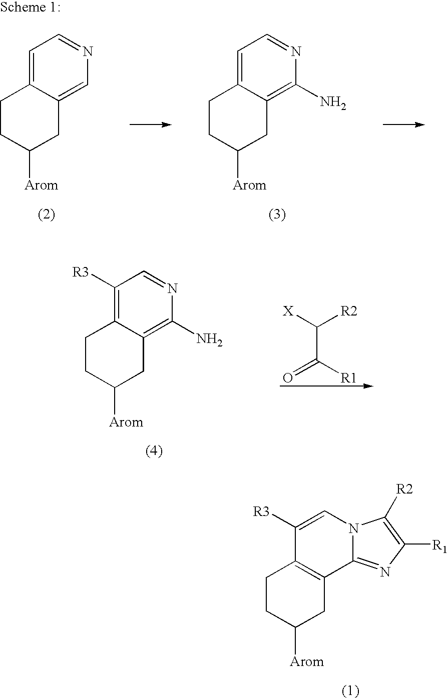

The derivatization, if any, of the compounds obtained according to the above scheme 1 (e.g. conversion of a group R3 into another group R3 or conversion of a group R2 into another group R2) is likewise carried out in a manner known to the expert. For example, if compounds where R3=—CO-1-4C-alkoxy, or where R3=—CO—NR31R32 are desired, an appropriate derivatization can be performed in a manner known to the expert (for example by metal-catalysed carbonylation of the corresponding halogen compound or conversion of an ester into an amide) at the stage of the compounds of formula 4 or more conveniently at a later point in time, for example conversion of a compound of the formula 1 into another compound of the formula 1.

If, according to Scheme 1, compounds of the formula 1 with R2=H are obtained, these compounds can be further transformed to a great variety of other compounds of the formula 1 (see Scheme 2). One example is the transformation via a Vilsmeier formylation to compounds of the formula 1 with R2=HC—(O)—, followed by further derivatization reactions, which are known to the expert (for example reduction of the aldehyde group, followed if desired by an etherification, or oxidation of the aldehyde group, followed by esterification or amide formation).

Another example for the derivatization of a compound of the formula 1 with R2=H is a halogenation reaction, for example a bromination reaction using a bromination reagent, like for example N-bromosuccinimide, to compounds of the formula 1 with R2=halogen, for example Br, followed by further transformations, for example by C—C-bond forming reactions, like for example Heck-, Suzuki- or Sonogashira-coupling reactions.

Another example for the derivatization of a compound of the formula 1 with R2=H is an aminoalkylation reaction, for example by electrophilic substitution with Eschenmoser's salt which leads to compounds of the formula 1 with, for example, R2=mono- or di-1-4C-alkylaminomethyl, for example dimethylaminomethylen. The resulting compounds can then be further derivatized, if desired, for example by treatment with an alkylation agent, e.g. methyl iodide, and subsequent nucleophilic substitution of the quartary ammonium group, e.g. vs. cyanide.

Still another access to further compounds of the formula 1 is, for example, offered by the transformation of compounds of the formula 1 with R2=H to compounds of the formula 1 with R2=NH₂. This transformation can be achieved for example in analogy to the reactions described in J. Med. Chem., 1989, 32, 1686 or by nitration of compounds of the formula 1 with R2=H and subsequent reduction of the nitro group. Further transformations by reactions known to the expert can then lead, if desired, to compounds of the formula 1 with R2=mono- or di-1-4C-alkylamino, 1-4C-alkylcarbonylamino, 1-4C-alkoxycarbonylamino or 1-4C-alkoxy-1-4C-alkoxycarbonylamino. Alternatively, compounds of the formula 1 with R2=NH₂ can be transformed into the corresponding diazonium salts. Further compounds of the formula 1, for example where R2 is e.g. hydroxy or 1-4-C-alkoxy, can then be obtained by substitution of the diazonium group via reactions known to the expert.

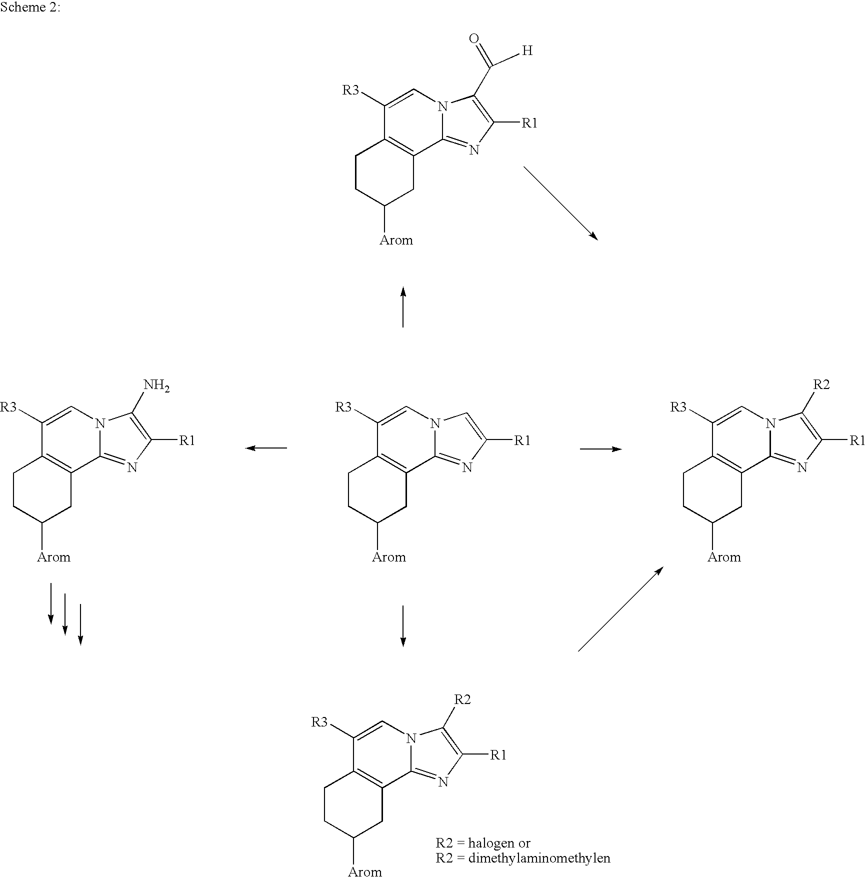

Compounds of the formula 2 can be prepared from the corresponding enamines of the formula 6 by Diels-Alder reaction with 1,2,4-triazine, in analogy to the reactions described for example in J. Org. Chem. 1981, 46, 2179-2182 (Scheme 3). The reagent 1,2,4-triazine can be prepared from commercially available starting materials, following for example the protocols described in J. Org. Chem. 1966, 31, 1720-22 and Synthesis 1974, 351-352. Enamines of the formula 6 can be obtained from the corresponding ketones of the formula 5 by condensation with a secondary amine, for example pyrrolidine, in the presence of dehydrating agents, for example titanium tetrachloride (Scheme 3) in analogy to the reactions described in J. Org. Chem. 1967, 32, 213-214.

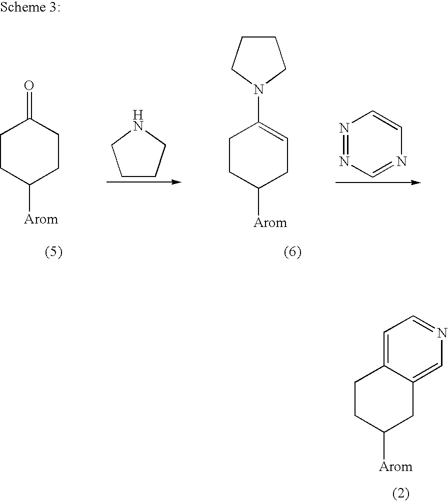

4-Aryl substituted cyclohexanones of the formula 5 are commercially available or can be obtained from commercially available starting materials, for example as depicted in Scheme 4: 4-Hydroxycyclohexanone (7) can be prepared from suitable precursors, for example as described in Org. Prep. Proced. 1969, 1(2), 127-129 or in Tetrahedron Asymm. 2003, 14(9), 1153-1160. Addition of Grignard reagents and subsequent oxidation (for example using chromium(VI) derivatives) leads to 4-aryl-4-hydroxycyclohexanones of the formula 8 (see for example J. Med. Chem. 1972, 15(12), 1235-1238). Compounds of the formula 5 can be obtained from these intermediates by elimination of water (for example by catalysis with trifluoroacetic acid) and subsequent reduction of the double bond (for example by Palladium-catalysed hydrogenation, for example as described in J. Med. Chem. 1972, 15(12), 1239-1243).

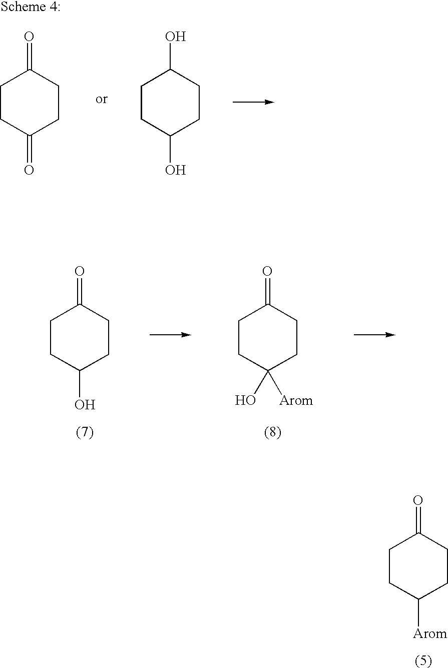

The examples below serve to illustrate the invention in more detail without limiting it. Further compounds of the formula 1, whose preparation is not described explicitly, can likewise be prepared in an analogous manner or in a manner known per se to the person skilled in the art, using customary process techniques. The compounds named expressly as examples, and the salts of these compounds, are preferred subject matter of the invention. The abbreviation v stands for volume. For the assignment of NMR signals, the following abbreviations are used: s (singlet), d (duplet), t (triplet), q (quartet), m_{c} (multiplet centred), b (broad). The following units are used: ml (millilitre), l (litre), mg (milligramme), g (gramme), mmol (millimol), N (normal), M (molar), min (minute), MHz (megahertz).

Furthermore the following abbreviations are used for the chemical substances indicated:

TBTU: O-benzotriazol-1-yl-N,N,N′,N′-tetramethyluronium tetrafluoroborate

THF: tetrahydrofuran

DMF: N,N-dimethylformamide

### EXAMPLES

I. Final Products

### 1. 2,3-Dimethyl-9-phenyl-7,8,9,10-tetrahydro-imidazo[2,1-a]isoquinoline-6-carboxylic acid ethyl ester

In a steel autoclave filled with argon, the hydrobromide salt of 6-bromo-2,3-dimethyl-9-phenyl-7,8,9,10-tetrahydro-imidazo[2,1-a]isoquinoline (example 1, 5.00 g, 11.5 mmol) was dissolved in dry ethanol (100 ml). After addition of triethylamine (7.5 ml, 53 mmol) a brown solution was obtained which was treated with palladium acetate (0.27 g, 1.2 mmol) and triphenylphosphine (0.40 g, 1.5 mmol). The autoclave was pressurized with carbon monoxide (6 bar) and heated to 115° C. The reaction mixture was kept for 18 hours at this temperature; cooled to room temperature, and poured onto a mixture of ice water (300 ml) and dichloromethane (300 ml). The phases were separated and the aqueous phase was extracted with dichloromethane (2×50 ml). The combined organic phases were washed with water (100 ml), dried over sodium sulfate and concentrated under reduced pressure. A brown solid (4.5 g) was obtained which was purified by flash chromatography [120 g of silica gel, eluant: petrol ether/ethyl acetate=6:4 (v/v)]. An almost colourless solid (melting point: 165-167° C.) was isolated (3.6 g, 90% yield) which was characterized as the pure title compound.

¹H-NMR (200 MHz, CDCl₃): δ=1.43 (t, 3H), 1.95 (m_{c}, 1H), 2.25 (m_{c}, 1H), 2.41, 2.43 (2 s, 6H), 3.01-3.39 (m, 4H), 3.55 (m_{c}, 1H), 4.40 (q, 2H), 7.27 (m_{c}), 8.42 (s, 1H).

### 2. 2,3-Dimethyl-9-phenyl-7,8,9,10-tetrahydro-imidazo[2,1-a]isoquinoline-6-carboxylic acid

A solution of 2,3-dimethyl-9-phenyl-7,8,9,10-tetrahydro-imidazo[2,1-a]isoquinoline-6-carboxylic acid ethyl ester (4.10 g, 11.7 mmol) in methanol (80 ml) was treated with an aqueous solution of potassium hydroxide (1.40 g, 25.0 mmol in 8 ml of water). The slightly yellow solution was heated to 60° C. for 3 hours. After the methanol had been removed under reduced pressure, the reaction mixture was diluted with water (30 ml) and extracted with ethyl acetate (2×20 ml). The organic phases were discarded and the aqueous phase (initial pH value: 11) was acidified by addition of hydrochloric acid (6 N, final pH value: 3). A suspension was obtained which was stirred for 2 hours at room temperature. A colourless solid was isolated by filtration, which was washed with portions of water (30 ml) and acetone (10 ml) and then dried in vacuo. The pure title compound (3.5 g, 93% yield) showed a melting point of 343-345° C. (decomposition).

¹H-NMR (200 MHz, DMSO-d₆+MeOH): δ=1.89 (m_{c}, 1H), 2.12 (m_{c}, 1H), 2.29 (s, 3H), 2.41 (s, 3H), 2.78-3.07 (m, 4H), 3.30 (m_{c}), 7.29 (m_{c}, 5H), 8.53 (s, 1H).

### 3. 2,3-Dimethyl-9-phenyl-7,8,9,10-tetrahydro-imidazo[2,1-a]isoquinoline-6-carboxylic acid dimethylamide

Under an argon atmosphere, a suspension of 2,3-dimethyl-9-phenyl-7,8,9,10-tetrahydro-imidazo[2,1-a]isoquinoline-6-carboxylic acid (500 mg, 1.56 mmol) in dry dichloromethane (8 ml) was treated with TBTU (550 mg, 1.71 mmol). The reaction mixture was heated to reflux for 1 hour. The suspension was cooled to room temperature and dimethylamine (0.80 ml of a 2 M solution in THF, 1.6 mmol) was added slowly. Stirring was continued for 3 hours at room temperature at which point saturated ammonium chloride solution (5 ml) was added to the yellow solution. The phases were separated and the aqueous phase was extracted with dichloromethane (6×4 ml). The combined organic phases were concentrated under reduced pressure. The residue was co-evaporated with dichloromethane (3×) and the foamy crude product (900 mg) was purified by flash chromatography [30 g of silica gel, eluant: dichloromethane/methanol=20:1 (v/v)] and subsequent washing with diethyl ether (8 ml). The pure title compound (390 mg, 72% yield) was isolated as a colourless solid (melting point: 239-241° C.).

¹H-NMR (200 MHz, CDCl₃): δ=1.99 (m_{c}, 1H), 2.22 (m_{c}, 1H), 2.37, 2.41 (2 s, 6H), 2.76 (bs, 2H), 2.92 (s, 3H), 3.07, 3.15 (m_{c}, s, 5H), 3.56 (m_{c}, 1H), 7.25 (m_{c}), 7.63 (s, 1H).

### 4. (2,3-Dimethyl-9-phenyl-7,8,9,10-tetrahydro-imidazo[2,1-a]isoquinolin-6-yl)-pyrrolidin-1-yl-methanone

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (10 ml) was treated with TBTU (500 mg, 1.56 mmol). The reaction mixture was heated to reflux for 1 hour. The suspension was cooled to room temperature and pyrrolidine (107 mg, 126 μl, 1.50 mmol) was added. Stirring was continued for 1 hour at room temperature at which point saturated ammonium chloride solution (10 ml) was added to the yellow solution. The phases were separated and the aqueous phase was extracted with dichloromethane (2×8 ml). The combined organic phases were washed with saturated sodium bicarbonate solution (10 ml), dried over sodium sulfate, and concentrated under reduced pressure. The residue (600 mg) was purified by washing with diethyl ether (10 ml). The pure title compound (450 mg, 86% yield) was isolated as a colourless solid (melting point 239-241° C., decomposition).

¹H-NMR (200 MHz, CDCl₃): δ=1.98 (m_{c}, 5H), 2.22 (m_{c}, 1H), 2.37, 2.41 (2 s, 6H), 2.80 (m_{c}, 2H), 3.07 (m_{c}, 2H), 3.24 (m_{c}, 2H), 3.53 (m_{c}, 1H), 3.67 (m_{c}, 2H), 7.24 (m_{c}), 7.66 (s, 1H).

### 5. 2,3-Dimethyl-9-phenyl-7,8,9,10-tetrahydro-imidazo[2,1-a]isoquinoline-6-carboxylic acid (2-hydroxy-ethyl)-amide

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (12 ml) was treated with TBTU (500 mg, 1.56 mmol). The reaction mixture was heated to reflux for 1 hour. The suspension was cooled to room temperature and 2-aminoethanol (101 mg, 100 μl, 1.66 mmol) was added. Stirring was continued for 2 hours at room temperature at which point saturated ammonium chloride solution (15 ml) and dichloromethane (30 ml) were added to the colourless suspension. The mixture was stirred for several minutes, the phases were separated, and the aqueous phase was extracted with dichloromethane (2×10 ml). The combined organic phases were washed with saturated sodium bicarbonate solution (20 ml) and water (2×20 ml), dried over sodium sulfate, and concentrated under reduced pressure. The residue (410 mg) was purified by washing with diethyl ether (15 ml). Thus the title compound (290 mg, 57% yield) was isolated as a colourless solid (melting point: 265-267° C.).

¹H-NMR (400 MHz, DMSO-d₆): δ=1.86 (m_{c}, 1H), 2.08 (m_{c}, 1H), 2.27 (s, 3H), 2.39 (s, 3H), 2.85, 3.00 (2 m_{c}, 4H), 3.25 (m_{c}), 3.54 (m_{c}, 2H), 4.72 (bt, 1H), 7.24 (m_{c}, 1H), 7.33 (m_{c}, 4H), 8.12 (s, 1H), 8.35 (t, 1H).

### 6. 2,3-Dimethyl-9-phenyl-7,8,9,10-tetrahydro-imidazo[2,1-a]isoquinoline-6-carboxylic acid (2-methoxy-ethyl)-amide

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (15 ml) was treated with TBTU (500 mg, 1.56 mmol). The reaction mixture was heated to reflux for 1 hour. The suspension was cooled to room temperature and 2-methoxyethylamine (112 mg, 130 μl, 1.50 mmol) was added. Stirring was continued for 2 hours at room temperature at which point the yellow solution was poured onto a mixture of saturated ammonium chloride solution (15 ml) and dichloromethane (30 ml). The mixture was stirred for several minutes, the phases were separated, and the aqueous phase was extracted with dichloromethane (2×10 ml). The combined organic phases were washed with saturated sodium bicarbonate solution (20 ml) and water (2×10 ml), dried over sodium sulfate, and concentrated under reduced pressure. The residue (470 mg) was purified by washing with diethyl ether (15 ml). Thus the title compound (390 mg, 74% yield) was isolated as a colourless solid (melting point: 208-210° C.).

¹H-NMR (400 MHz, DMSO-d₆): δ=1.87 (m_{c}, 1H), 2.09 (m_{c}, 1H), 2.27 (s, 3H), 2.38 (s, 3H), 2.85, 3.00 (2 m_{c}, 4H), 3.27 (m_{c}), 3.40 (m_{c}, 2H), 3.46 (m_{c}, 2H), 7.24 (m_{c}, 1H), 7.33 (m_{c}, 4H), 8.07 (s, 1H), 8.43 (t, 1H).

### 7. (2,3-Dimethyl-9-phenyl-7,8,9,10-tetrahydro-imidazo[2,1-a]isoquinolin-6-yl)-morpholin-4-yl-methanone

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (15 ml) was treated with TBTU (500 mg, 1.56 mmol). The reaction mixture was heated to reflux for 1 hour. The suspension was cooled to room temperature and morpholine (130 mg, 130 μl, 1.49 mmol) was added. Stirring was continued for 1 hour at room temperature at which point the yellow solution was poured onto a mixture of saturated ammonium chloride solution (10 ml) and dichloromethane (20 ml). The mixture was stirred for several minutes, the phases were separated, and the aqueous phase was extracted with dichloromethane (2×10 ml). The combined organic phases were washed with saturated sodium bicarbonate solution (15 ml) and water (10 ml), dried over sodium sulfate, and concentrated under reduced pressure. The residue (600 mg) was purified by washing with diethyl ether (10 ml). The pure title compound (440 mg, 81% yield) was isolated as a colourless solid (melting point: 184-186° C.).

¹H-NMR (400 MHz, DMSO-d₆): δ=1.92 (m_{c}, 1H), 2.09 (m_{c}, 1H), 2.27 (s, 3H), 2.37 (s, 3H), 2.77, 2.87 (bs, m_{c}, 3H), 3.03 (m_{c}, 1H), 3.15-3.45 (bm), 3.45-3.80 (bm, 6H), 7.24 (m_{c}, 1H), 7.34 (m_{c}, 4H), 8.05 (s, 1H).

### 8. 2,3-Dimethyl-9-phenyl-7,8,9,10-tetrahydro-imidazo[2,1-a]isoquinoline-6-carboxylic acid methylamide

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (15 ml) was treated with TBTU (500 mg, 1.56 mmol). The reaction mixture was heated to reflux for 1 hour. The suspension was cooled to room temperature and methylamine (750 μl of a 2 M solution in THF, 1.50 mmol) was added slowly. Stirring was continued for 1 hour at room temperature at which point the yellow solution was poured onto a mixture of saturated ammonium chloride solution (15 ml) and dichloromethane (30 ml). The mixture was stirred for several minutes, the phases were separated, and the aqueous phase was extracted with dichloromethane (2×10 ml). The combined organic phases were washed with saturated sodium bicarbonate solution (15 ml) and water (20 ml), dried over sodium sulfate, and concentrated under reduced pressure. The residue (400 mg) was purified by washing with diethyl ether (15 ml). The title compound (260 mg, 56% yield) was isolated as a colourless solid (melting point: 266-268° C.).

¹H-NMR (400 MHz, DMSO-d₆): δ=1.86 (m_{c}, 1H), 2.08 (m_{c}, 1H), 2.27 (s, 3H), 2.38 (s, 3H), 2.78 (d, 3H), 2.85, 3.00 (2 m_{c}, 4H), 3.30 (m_{c}), 7.24 (m_{c}, 1H), 7.34 (m_{c}, 4H), 8.11 (s, 1H), 8.30 (q, 1H).

### 9. 2,3-Dimethyl-9-phenyl-7,8,9,10-tetrahydro-imidazo[2,1-a]isoquinoline-6-carboxylic acid amide

Under an argon atmosphere, a suspension of 2,3-dimethyl-9-phenyl-7,8,9,10-tetrahydro-imidazo[2,1-a]isoquinoline-6-carboxylic acid (example 2, 450 mg, 1.40 mmol) in dry dichloromethane (18 ml) was treated with TBTU (500 mg, 1.56 mmol). The reaction mixture was heated to reflux for 1 hour. The well-stirred suspension was cooled to room temperature and was saturated with ammonia gas for 1 hour. The colourless suspension was poured onto a mixture of saturated ammonium chloride solution (20 ml) and dichloromethane (30 ml). The mixture was stirred for several minutes and the pH (initial value: 10) was adjusted to 6 by addition of 6 N hydrochloric acid. The phases were separated, and the aqueous phase was extracted with dichloromethane (20 ml). The combined organic phases were concentrated under reduced pressure. The oily residue was treated with acetone (10 ml) at which point crystallization occurred. The solid was removed by filtration and washed with acetone (8 ml) and diethyl ether (10 ml). After drying in vacuo, 290 mg (65% yield) of the pure title compound were obtained in form of a colourless solid (melting point: 298-300° C.).

¹H-NMR (400 MHz, DMSO-d₆): δ=1.94 (m_{c}, 1H), 2.17 (m_{c}, 1H), 2.44 (s, 3H), 2.49 (s), 2.98, 3.11 (2 m_{c}, 4H), 3.36 (m_{c}), 7.27 (m_{c}, 1H), 7.38 (m_{c}, 4H), 7.83 (s, 1H), 8.21 (s, 1H), 8.67 (s, 1H).

### 10. (2,3-Dimethyl-9-phenyl-7,8,9,10-tetrahydro-imidazo[2,1-a]isoquinolin-6-yl)-methanol

In a flame-dried flask filled with argon, 2,3-dimethyl-9-phenyl-7,8,9,10-tetrahydro-imidazo[2,1-a]isoquinoline-6-carboxylic acid ethyl ester (example 1, 2.50 g, 7.2 mmol) was dissolved in dry THF (30 ml). At room temperature, lithium aluminium hydride (0.40 g, 10.5 mmol) was added in small portions. Stirring was continued for 1 hour at room temperature and the reaction mixture was quenched by addition of water (0.5 ml), sodium hydroxide solution (15 weight-%, 0.5 ml), and more water (1.5 ml). The grey suspension was stirred for 20 minutes at room temperature and was filtered. The filter cake was washed with THF (3×10 ml) and was then suspended in a mixture of chloroform (30 ml) and methanol (15 ml). The resulting slurry was stirred for 1 hour at room temperature. Insoluble material was removed by filtration and the filter cake was washed with chloroform (10 ml) and methanol (10 ml). The combined filtrates were evaporated to dryness. The residue, 980 mg of a colourless solid, was dried in vacuo and characterized as the title compound (melting point: 290-292° C., 44% yield).

¹H-NMR (200 MHz, DMSO-d₆+MeOH): δ=1.95 (m_{c}, 1H), 2.13 (m_{c}, 1H), 2.27 (s, 3H), 2.37 (s, 3H), 2.89 (m_{c}, 4H), 3.30 (m_{c}, 1H), 4.54 (s, 2H), 7.32 (m_{c}, 5H), 7.92 (s, 1H).

### 11. 6-Methoxymethyl-2,3-dimethyl-9-phenyl-7,8,9,10-tetrahydro-imidazo[2,1]isoquinoline

6-Chloromethyl-2,3-dimethyl-9-phenyl-7,8,9,10-tetrahydro-imidazo[2,1-a]isoquinoline (example L, 500 mg, 1.54 mmol) was suspended in dry methanol (12 ml). After addition of sodium methylate (solution: 30 weight-% in methanol, 0.56 ml, 3.0 mmol) the reaction mixture was heated to 60° C. Within a period of 90 minutes a yellow solution was formed, which was cooled to room temperature and poured onto a mixture of saturated ammonium chloride solution (20 ml) and dichloromethane (50 ml). The phases were separated and the aqueous phase was extracted with dichloromethane (3×5 ml). The combined organic phases were washed with water (15 ml), dried over sodium sulfate, and concentrated under reduced pressure. An oily residue (520 mg) was isolated which was purified by flash chromatography [20 g of silica gel, eluant: dichloromethane/methanol=20:1 (v/v)]. Evaporation of the corresponding fractions afforded an oily residue (380 mg), which was crystallized from diethyl ether (3 ml). The title compound was isolated by filtration, washed with diethyl ether (1 ml), and dried in vacuo (210 mg of a colourless solid, 44% yield, melting point: 113-114° C.).

¹H-NMR (200 MHz, DMSO-d₆): δ=1.90 (m_{c}, 1H), 2.11 (m_{c}, 1H), 2.26 (s, 3H), 2.36 (s, 3H), 2.88 (m_{c}, 4H), 3.25 (m_{c}, 1H), 3.31 (s, 3H), 4.44 (s, 2H), 7.29 (m_{c}, 5H), 7.98 (s, 1H).

### 12. 2-Methyl-9-phenyl-7,8,9,10-tetrahydro-imidazo[2,1-a]isoquinoline-6-carboxylic acid dimethylamide

In a steel autoclave filled with argon, the hydrochloride salt of 6-bromo-2-methyl-9-phenyl-7,8,9,10-tetrahydro-imidazo[2,1-a]isoquinoline (example J, 2.80 g, 7.4 mmol) was suspended in dry THF (10 ml). After addition of dimethylamine (38.0 ml of a 2 M solution in THF, 76 mmol), palladium acetate (0.30 g, 1.3 mmol), triphenylphosphine (1.10 g, 4.2 mmol), and triethylamine (2.0 ml, 14 mmol), the autoclave was pressurized with carbon monoxide (6 bar) and heated to 120° C. The reaction mixture was kept for 19 hours at this temperature, cooled to room temperature, and poured onto a mixture of saturated ammonium chloride solution (80 ml) and ethyl acetate (80 ml). The phases were separated and the aqueous phase was extracted with ethyl acetate (3×20 ml) The combined organic phases were washed with saturated ammonium chloride solution (2×50 ml) and water (2×50 ml), dried over sodium sulfate, and concentrated under reduced pressure. A brown solid (6 g) was obtained which was purified by flash chromatography [100 g of silica gel, eluant: ethyl acetate/methanol=100:3 (v/v)] and subsequent washing with diethyl ether. This afforded the pure title compound (1.7 g of a colourless solid, 69% yield, melting point: 215-217° C.).

¹H-NMR (200 MHz, CDCl₃): δ=1.98 (m_{c}, 1H), 2.21 (m_{c}, 1H), 2.44 (s, 3H), 2.76 (bs, 2H), 2.93, 3.07, 3.14 (s, m_{c}, s, 8H), 3.55 (m_{c}, 1H), 7.25 (m_{c}), 7.85 (s, 1H).

### 13. 3-Bromo-2-methyl-9-phenyl-7,8,9,10-tetrahydro-imidazo[2,1-a]isoquinoline-6-carboxylic acid dimethylamide

2-Methyl-9-phenyl-7,8,9,10-tetrahydro-imidazo[2,1-a]isoquinoline-6-carboxylic acid dimethylamide (350 mg, 1.05 mmol) was dissolved in dry dichloromethane (8 ml). The solution was cooled to −75° C. and a suspension of N-bromosuccinimide (195 mg, 1.10 mmol) in dichloromethane (6 ml) was added over a period of 10 minutes. The reaction mixture was stirred for 1 hour at −75° C. and was then quenched by addition of saturated sodium bicarbonate solution (8 ml). The phases were separated and the aqueous phase was extracted with dichloromethane (2×5 ml). The combined organic phases were washed with water (10 ml), dried over sodium sulfate, and concentrated under reduced pressure. The foamy, colourless residue (500 mg) was crystallized from diethyl ether (10 ml). The pure title compound (385 mg, 89% yield) was isolated as a colourless solid (melting point: 166-168° C.).

¹H-NMR (200 MHz, CDCl₃): δ=1.99 (m_{c}, 1H), 2.23 (m_{c}, 1H), 2.45 (s, 3H), 2.77 (bs, 2H), 2.93 (s, 3H), 3.06 (m_{c}, 2H), 3.16 (s, 3H), 3.55 (m_{c}, 1H), 7.28 (m_{c}), 7.86 (s, 1H).

### 14. 3-Hydroxymethyl-2-methyl-9-phenyl-7,8,9,10-tetrahydro-imidazo[2,1-a]isoquinoline-6-carboxylic acid dimethylamide

3-Formyl-2-methyl-9-phenyl-7,8,9,10-tetrahydro-imidazo[2,1-a]isoquinoline-6-carboxylic acid dimethylamide (example K, 300 mg, 0.83 mmol) was dissolved in dry methanol (10 ml). Sodium borohydride (40 mg, 1.06 mmol) was added in small portions. A clear solution was obtained which was stirred for 30 minutes at room temperature and then poured onto a mixture of saturated ammonium chloride solution (10 ml) and dichloromethane (20 ml). The phases were separated and the aqueous phase was extracted with dichloromethane (3×5 ml). The combined organic phases were washed with saturated ammonium chloride solution (11 ml) and water (10 ml), dried over sodium sulfate, and concentrated under reduced pressure. The colourless residue (300 mg) was purified by flash chromatography [30 g of silica gel, eluant: dichloromethane/methanol=100:3 (v/v)]. The pure title compound (190 mg, 63% yield) was isolated as a colourless solid (melting point: 385-388° C.).

¹H-NMR (200 MHz, CDCl₃): δ=2.07 (m_{c}, 1H), 2.23 (m_{c}, 1H), 2.39 (s, 3H), 2.77 (bs, 2H), 2.92 (s, 3H), 3.05 (m_{c}, 2H), 3.14 (s, 3H), 3.52 (m_{c}, 1H), 4.90 (s, 2H), 7.26 (m_{c}), 8.02 (s, 1H).

II. Starting Compounds and Intermediates

### A. 1-(4-Phenyl-cyclohex-1-enyl)-pyrrolidine

In a flame-dried flask filled with argon, 4-phenylcyclohexanone (12.5 g, 72 mmol) was suspended in dry n-hexane (250 ml) and a solution of pyrrolidine (25.0 g, 350 mmol) in n-hexane (30 ml) was added. The clear solution was cooled to 0° C. and a solution of titanium tetrachloride (6.8 g, 36 mmol) in n-hexane (50 ml) was added drop-wise over a period of 1 hour at which point a green-white precipitate was formed. The reaction mixture was allowed to come to room temperature and stirring was continued for 3 hours. The precipitate was removed by filtration and was washed with n-hexane (2×20 ml). The filtrates were concentrated under reduced pressure. An oily residue (13.5 g) was isolated which was characterized by ¹H-NMR-spectroscopy. The sample contained 81 weight-% of 1-(4-phenyl-cyclohex-1-enyl)-pyrrolidine (10.9 g, 68% yield), 15 weight-% of 4-phenylcyclohexanone, and 4 weight-% of pyrrolidine.

¹H-NMR (200 MHz, DMSO-d₆): δ=1.78, 2.25, 2.95 (3 m_{c}), 4.18 (m_{c}, 1H), 7.25 (m_{c}).

### B. 7-Phenyl-5,6,7,8-tetrahydro-isoquinoline

In a flame-dried flask filled with argon, 1,2,4-triazine (20.0 g, 0.25 mol) was dissolved in dry chloroform (200 ml). A solution of 1-(4-phenyl-cyclohex-1-enyl)-pyrrolidine (92.0 g, 75 weight-%, 0.30 mol) in chloroform (180 ml) was added slowly. The red solution was heated to 60° C. for 18 hours. The reaction mixture was cooled to 0° C. and was then poured onto saturated ammonium chloride solution (300 ml). Stirring was continued for several minutes and the phases were separated. The aqueous phase was extracted with chloroform (2×50 ml). The combined organic phases were washed with saturated ammonium chloride solution (2×80 ml) and water (2×80 ml), dried over sodium sulfate and evaporated to dryness. The residue (130 g of a brown oil) was purified by flash chromatography [500 g of silica gel, eluant: ethyl acetate/petrol ether=1:1 (v/v)]. A brownish oil (44.0 g, 84% yield) was isolated which was characterized as the pure title compound.

¹H-NMR (200 MHz, CDCl₃): δ=1.92 (m_{c}, 1H), 2.15 (m_{c}, 1H), 2.93 (m_{c}, 5H), 7.02 (d, 1H), 7.28 (m_{c}), 8.31 (d, 1H), 8.33 (s, 1H).

### C. 1-[4-(4-Fluorophenyl)-cyclohex-1-enyl]-pyrrolidine

In a flame-dried flask filled with argon and equipped with a mechanical stirrer, 4-(4-fluorophenyl)-cyclohexanone (6.0 g, 31 mmol) was suspended in dry n-hexane (120 ml) and pyrrolidine (11.0 g, 155 mmol) was added. The clear solution was cooled to 0° C. and a solution of titanium tetrachloride (3.0 g, 16 mmol) in n-hexane (20 ml) was added drop-wise over a period of 30 minutes at which point a green-white suspension was formed. The reaction mixture was allowed to come to room temperature and stirring was continued for 15 hours. The precipitate was removed by filtration and was washed with n-hexane (3×30 ml). The filtrates were concentrated under reduced pressure. The residue was dried in vacuo. The pure title compound was obtained as a yellow solid (6.0 g, 79% yield, melting point: 58-60° C.).

¹H-NMR (200 MHz, CDCl₃): δ=1.87 (m_{c}, 6H), 2.39 (m_{c}, 4H), 2.75 (m_{c}, 1H), 3.00 (m_{c}, 4H), 4.35 (bs, 1H), 6.99 (m_{c}, 2H), 7.20 (m_{c}, 2H).

### D. 7-(4-Fluorophenyl)-5,6,7,8-tetrahydro-isoquinoline

In a flame-dried flask filled with argon, 1,2,4-triazine (2.4 g, 30 mmol) was dissolved in dry chloroform (25 ml). A solution of 1-[4-(4-fluorophenyl)-cyclohex-1-enyl]-pyrrolidine (5.9 g, 24 mmol) in chloroform (20 ml) was added slowly. The red solution was heated to 60° C. for 18 hours. The reaction mixture was cooled to room temperature and was then poured onto a stirred cold mixture of saturated ammonium chloride solution (30 ml) and dichloromethane (30 ml) The phases were separated and the aqueous phase was extracted with dichloromethane (3×10 ml). The combined organic phases were washed with saturated ammonium chloride solution (20 ml) and water (20 ml), dried over sodium sulfate, and evaporated to dryness. The residue (8 g of a dark oil) was purified by flash chromatography [60 g of silica gel, eluant: dichloromethane]. Evaporation of the corresponding fractions afforded the title compound (2.1 g of a brown oil, 38% yield).

¹H-NMR (200 MHz, DMSO-d₆): δ=1.86 (m_{c}, 1H), 2.00 (m_{c}, 1H), 2.84 (m_{c}, 3H), 2.98 (m_{c}, 2H), 7.16 (m_{c}, 3H), 7.37 (m_{c}, 2H), 8.26 (d, 1H), 8.31 (s, 1H).

### E. 1-[4-(4-Benzyloxyphenyl)cyclohex-1 enyl]-pyrrolidine

In a flame-dried flask filled with argon, 4-(4-benzyloxyphenyl)-cyclohexanone (6.0 g, 21 mmol) was suspended in dry toluene (130 ml) and pyrrolidine (7.6 g, 107 mmol) was added. The mixture was stirred at room temperature until a clear solution was obtained (approximately 1 hour). A solution of titanium tetrachloride (2.1 g, 11 mmol) in toluene (15 ml) was added drop-wise over a period of 30 minutes at which point a dark suspension was formed. The reaction mixture was stirred for 18 hours at room temperature. The precipitate was removed by filtration and was washed with toluene (100 ml). The filtrates were concentrated under reduced pressure. A yellow solid (7.2 g) was isolated which was characterized by ¹H-NMR spectroscopy. The sample contained 53 weight-% of 1-[4-(4-benzyloxyphenyl)-cyclohex-1-enyl]-pyrrolidine (3.8 g, 53% yield), 37 weight-% of 4-(4-benzyloxyphenyl)-cyclohexanone, and 10 weight-% of pyrrolidine.

¹H-NMR (200 MHz, CDCl₃): δ=1.89 (m_{c}), 2.10-2.80 (m), 3.05 (m_{c}), 4.34 (bs, 1H), 5.04 (s), 6.93 (m_{c}), 7.16 (m_{c}), 7.39 (m_{c}).

### F. 7-(4-Benyloxyphenyl)-5,6,7,8-tetrahydro-isoquinoline

In a flame-dried flask filled with argon, 1,2,4-triazine (1.10 g, 13.6 mmol) was dissolved in dry chloroform (10 ml). A solution of 1-[4-(4-benzyloxyphenyl)-cyclohex-1-enyl]-pyrrolidine (7.00 g, 53 weight-%, 11.1 mmol) in chloroform (35 ml) was added slowly. The yellow solution was heated to 60° C. for 1 day. The reaction mixture was cooled to room temperature and was then poured onto a cold mixture of saturated ammonium chloride solution (30 ml) and dichloromethane (20 ml). The phases were separated and the aqueous phase was extracted with dichloromethane (2×15 ml). The combined organic phases were washed with water (2×30 ml), dried over sodium sulfate, and evaporated to dryness. The residue (8 g of a dark oil) was purified by flash chromatography [80 g of silica gel, eluant: dichloromethane]. Evaporation of the corresponding fractions afforded the title compound (3.6 g of a brown solid, quant.).

¹H-NMR (200 MHz, CDCl₃): δ=1.93 (m_{c}, 1H), 2.14 (m_{c}, 1H), 2.92 (m_{c}, 5H), 5.07 (s, 2H), 6.96 (m_{c}, 2H), 7.06 (d, 1H), 7.19 (m_{c}, 2H), 7.39 (m_{c}, 5H), 8.25 (d, 1H), 8.28 (s, 1H).

### G. 7-Phenyl-5,6,7,8-tetrahydro-isoquinolin-1-yl-amine

(a) Microwave Synthesis: In a microwave reaction vessel, which had been filled with argon, 7-phenyl-5,6,7,8-tetrahydro-isoquinoline (example B, 0.25 g, 1.2 mmol) was dissolved in dimethylaniline (6.3 ml). Under an argon atmosphere, sodium amide pellets (0.16 g, 4.1 mmol) were crushed and added to the reaction mixture. The vessel was closed and was heated to 180° C. in a microwave oven (Emry's optimiser, Personal Chemistry, power input: 20-25 watt, pressure: 7.1-8.5 bar) for 12 hours. The reaction mixture was poured onto a cold mixture of saturated ammonium chloride solution (30 ml) and ethyl acetate (30 ml). Stirring was continued for several minutes. The phases were separated and the aqueous phase was extracted with ethyl acetate (2×15 ml). The combined organic phases were washed with saturated ammonium chloride solution (2×15 ml) and water (2×20 ml), dried over sodium sulfate and evaporated to dryness. The obtained brown liquid was purified by flash chromatography (15 g of silica gel 15-25 μm, eluant: dichloromethane) to give 160 mg (59% yield) of the title compound. The ¹H-NMR spectrum of the isolated brown solid showed characteristic signals of the title compound and traces of impurities.

(b) Thermal Synthesis: In a steel-autoclave filled with argon, 7-phenyl-5,6,7,8-tetrahydro-isoquinoline (example B, 5.00 g, 23.9 mmol) was dissolved in tetralin (50 ml), which had been degassed with argon. Crushed sodium amide pellets (2.8 g, 72 mmol) were added and the resulting suspension was heated for 18 hours to 220° C. The dark-brown reaction mixture was cooled to room temperature and poured onto a mixture of saturated ammonium chloride solution (50 ml) and dichloromethane (80 ml). The phases were separated and the aqueous phase was extracted with dichloromethane (2×20 ml). The combined organic phases were washed with saturated ammonium chloride solution (50 ml) and water (2×30 ml), dried over sodium sulfate, and concentrated under reduced pressure. The obtained dark-brown residue (60 g) contained tetralin, which was removed by flash chromatography (700 g of silica gel, eluant: dichloromethane). After exchange of the eluant [diethyl ether/triethylamin=20:1 (v/v)], 2.6 g of the title compound (48% yield, yellow-brown solid, melting point 124-125° C.) and 0.85 g of its regioisomer 7-phenyl-5,6,7,8-tetrahydro-isoquinolin-3-yl-amine (16% yield, brown solid containing impurities) were eluted.

¹H-NMR (200 MHz, CDCl₃): δ=1.91 (m_{c}, 1H), 2.12 (m_{c}, 1H), 2.45 (m_{c}, 1H), 2.75 (m_{c}, 3H), 3.02 (m_{c}, 1H), 4.32 (bs, 2H), 6.50 (d, 1H), 7.31 (m_{c}), 7.85 (d, 1H).

### H. 4-Bromo-7-phenyl-5,6,7,8-tetrahydro-isoquinolin-1-yl-amine

In a flask filled with argon, 7-phenyl-5,6,7,8-tetrahydro-isoquinolin-1-yl-amine (4.20 g, 18.7 mmol) was dissolved in dry acetonitrile (60 ml). N-Bromosuccinimide (3.50 g, 19.7 mmol) was added in small portions over a period of 20 minutes. The slightly red-coloured reaction mixture was stirred for 30 minutes at room temperature. The obtained suspension was poured onto a mixture of ice (80 g), saturated ammonium chloride solution (50 ml) and ethyl acetate (120 ml). The phases were separated and the aqueous phase was extracted with ethyl acetate (50 ml). The combined organic phases were washed with water (80 ml), dried over sodium sulfate, and concentrated under reduced pressure. Thus, 5.50 g of the title compound (97% yield) were isolated. Traces of impurities (succinimide) were visible in the ¹H-NMR spectrum of the brown solid (melting point 122-125° C.).

¹H-NMR (200 MHz, CDCl₃): δ=1.92 (m_{c}, 1H), 2.19 (m_{c}, 1H), 2.47 (m_{c}, 1H), 2.71 (m_{c}, overlay with succinimide: 2.75), 2.98 (m_{c}, 2H), 4.50 (bs, 2H), 7.33 (m_{c}), 8.02 (s, 1H).

### I. 6-Bromo-2,3-dimethyl-9-phenyl-7,8,9,10-tetrahydro-imidazo[2,1-a]isoquinoline, hydro-bromide salt

In a flask filled with argon, 4-bromo-7-phenyl-5,6,7,8-tetrahydro-isoquinolin-1-yl-amine (6.40 g, 21.1 mmol) was dissolved in dry THF (120 ml). 3-Bromobutanone (4.2 ml, 6.0 g, 40 mmol) was added and the reaction mixture was heated to reflux for 90 hours. A precipitate was formed which was removed by filtration, washed with THF, and dried in vacuo (3.0 g of the pure title compound, colourless solid, melting point: 263-265° C.). The mother liquor was treated with another portion of 3-bromobutanone (3.0 ml, 4.3 g, 29 mmol) and was refluxed for another 75 hours. A second crop of crystals was obtained, which were isolated and purified as described above (3.0 g of the pure title compound, colourless solid, melting point: 263-265° C.). Thus, a total amount of 6.0 g (66% yield) of the title compound was obtained.

¹H-NMR (200 MHz, DMSO-d₆): δ=2.00 (m_{c}, 1H), 2.20 (m_{c}, 1H), 2.43 (s, 3H), 2.50 (s), 3.00 (m_{c}, 4H), 3.24 (m_{c}), 7.32 (m_{c}, 5H), 8.96 (s, 1H).

### J. 6-Bromo-2-methyl-9-phenyl-7,8,9,10-tetrahydro-imidazo[2,1-a]isoquinoline, hydrochloride salt

In a flask filled with argon, 4-bromo-7-phenyl-5,6,7,8-tetrahydro-isoquinolin-1-yl-amine (example H, 2.90 g, 9.6 mmol) was dissolved in dry THF (25 ml). Chloroacetone (1.30 ml, 1.51 g, 16.3 mmol) was added and the reaction mixture was heated to reflux for 2.5 days. The red-brown suspension was cooled to room temperature. The precipitate was isolated by filtration and was washed with THF (10 ml) and diethyl ether (10 ml). Thus, the pure title compound (2.55 g, 70% yield) was obtained as a colourless solid (melting point: 273-275° C.). The mother liquor was treated with another portion of chloroacetone (0.60 ml, 0.70 g, 7.5 mmol) and was refluxed for 50 hours. The precipitate formed was isolated by filtration and purified as described above. Another portion (0.40 g, 1.1 mmol, 11% yield) of the pure title compound was isolated (melting point: 273-275° C., overall yield: 81%).

¹H-NMR (200 MHz, DMSO-d₆): δ=2.02 (m_{c}, 1H), 2.22 (m_{c}, 1H), 2.48 (s), 3.03 (m_{c}, 4H), 3.36 (m_{c}), 7.33 (m_{c}, 5H), 7.99 (s, 1H), 9.19 (s, 1H).

### K. 3-Formyl-2-methyl-9-phenyl-7,8,9,10-tetrahydro-imidazo[2,1-a]isoquinoline-6-carboxylic acid dimethylamide

Under an argon atmosphere at a temperature of 0° C., phosphorus oxychloride (0.33 ml, 0.54 g, 3.5 mmol) was added drop wise to dry DMF (3.5 ml). After the pink solution had been stirred for 90 minutes at room temperature, a solution of 2-methyl-9-phenyl-7,8,9,10-tetrahydro-imidazo[2,1-a]isoquinoline-6-carboxylic acid dimethylamide (example 12, 800 mg, 2.4 mmol) in dry DMF (11 ml) was added slowly. A red solution was obtained which was stirred for 1 hour at room temperature and for 3 hours at 60° C. The reaction mixture was cooled to room temperature, poured onto a mixture of ice water (20 ml) and dichloromethane (20 ml), and neutralized by addition of 25% aqueous ammonia solution. The phases were separated and the aqueous phase was extracted with dichloromethane (3×10 ml). The combined organic phases were washed with water (4×10 ml), dried over sodium sulfate, and concentrated under reduced pressure. The colourless residue (1.1 g) was purified by flash chromatography [40 g of silica gel, eluant: ethyl acetate/methanol=20:1 (v/v)]. The composition of the obtained slightly red solid (680 mg, 78%, melting point: 205-207° C.) was determined by ¹H-NMR spectroscopy. The sample consisted of the title compound along with 9 weight-% of untransformed starting material (2-methyl-9-phenyl-7,8,9,10-tetrahydro-imidazo[2,1-a]isoquinoline-6-carboxylic acid dimethylamide).

¹H-NMR (200 MHz, CDCl₃): δ=2.00 (m_{c}, 1H), 2.25 (m_{c}, 1H), 2.70 (s, 3H), 2.92, 2.95, 3.00, 3.17 (m_{c}, s, m_{c}, s, 10H), 3.57 (m_{c}, 1H), 7.31 (m_{c}), 9.30 (s, 1H), 9.98 (s, 1H).

### L. 6-Chloromethyl-2,3-dimethyl-9-phenyl-7,8,9,10-tetrahydro-imidazo[2,1-a]isoquinoline

At room temperature, thionyl chloride (0.23 ml, 0.38 g, 3.2 mmol) was added slowly to a suspension of (2,3-dimethyl-9-phenyl-7,8,9,10-tetrahydro-imidazo[2,1-a]isoquinolin-6-yl)-methanol (example 10, 0.95 g, 3.1 mmol) in dry dichloromethane (25 ml). The resulting solution was stirred for 1 hour at room temperature, cooled to 0° C., and treated slowly with a mixture of saturated sodium bicarbonate solution (8 ml) and water (5 ml). The cooling bath was removed and the biphasic mixture was stirred for 10 minutes. The phases were separated and the aqueous phase was extracted with dichloromethane (10 ml). The combined organic phases were washed with water (10 ml), dried over sodium sulfate, and the solvent was evaporated under reduced pressure. A brown solid remained which was suspended in diethyl ether (12 ml). The title compound was isolated by filtration, washed with diethyl ether (5 ml), and dried in vacuo (880 mg, 87% yield, melting point: 155-157° C.).

¹H-NMR (200 MHz, DMSO-d₆): δ=1.95 (m_{c}, 1H), 2.14 (m_{c}, 1H), 2.27 (s, 3H), 2.37 (s, 3H), 2.92 (m_{c}, 4H), 3.27 (m_{c}, 1H), 4.90 (m_{c}, 2H), 7.29 (m_{c}, 5H), 8.27 (s, 1H).

### COMMERCIAL UTILITY

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDs), which are known to have a certain ulcerogenic potency. In addition, the compounds of formula 1 are suited for a free or fixed combination with motility-modifying drugs.

### Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds according to the invention on the pentagastrin-stimulated acid secretion of the perfused rat stomach after intraduodenal administration in vivo is shown.

TABLE A

| | Dose | |
|---|---|---|
| | (μmol/kg) | Inhibition of acid secretion |
| No. | i.d. | (%) |
| 3 | 3 | >40 |
| 4 | 3 | >40 |
| 6 | 3 | >40 |
| 7 | 3 | >40 |
| 8 | 3 | >40 |
| 11 | 3 | >40 |

Methodology

The abdomen anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the formula 1 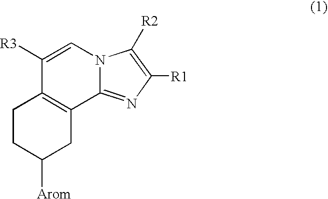
in which
R1 is hydrogen, 1-4C-alkyl, 3-7C-cycloalkyl, 1-4C-alkoxy-1-4C-alkyl, 1-4C-alkoxycarbonyl, 2-4C-alkenyl, 2-4C-alkinyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, hydroxy-3-4-C-alkenyl, hydroxy-3-4C-alkinyl, 1-4C-alkoxycarbonyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R4 is hydrogen, 1-4C-alkyl, hydroxy-1-4C-alkyl, 1-4C-alkoxy, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxyl,
R6 is hydrogen, 1-4C-alkyl or halogen and
R7 is hydrogen, 1-4C-alkyl or halogen,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
or a salt thereof.

2. A compound of the formula 1 as claimed in claim 1, in which
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl radical,
where
R4 is hydrogen, 1-4C-alkyl, hydroxy-1-4C-alkyl, 1-4C alkoxy, 1-4C-alkoxycarbonyl, carboxy-1-4C-alkyl, halogen, hydroxyl or trifluoromethyl,
R5 is hydrogen, 1-4C-alkyl, halogen, or hydroxyl and
R6 is hydrogen or 1-4C-alkyl,
or a salt thereof.

3. A compound of the formula 1 as claimed in claim 1, in which
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5- and R6-substituted phenyl radical,
where
R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl or trifluoromethyl,
R5 is hydrogen, 1-4C-alkyl, halogen, or hydroxyl and
R6 is hydrogen or 1-4C-alkyl,
or a salt thereof.

4. A compound of the formula 1 as claimed in claim 1, in which
R1 is 1-4C-alkyl or 3-7C-cycloalkyl,
R2 is hydrogen, 1-4C-alkyl, 3-7C-cycloalkyl, halogen, 2-4C-alkenyl, 2-4C-alkynyl, hydroxy-1-4C-alkyl, 1-4C-alkoxycarbonyl, fluoro-1-4C-alkyl, cyanomethyl, carboxyl, 1-4C-alkylcarbonyl, or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is phenyl,
or a salt thereof.

5. A compound of the formula 1 as claimed in claim 1, in which
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5- and R6-substituted phenyl, pyrrolyl, furanyl (furyl), thiophenyl (thienyl) or pyridinyl radical,
where
R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl or trifluoromethyl,
R5 is hydrogen, 1-4C-alkyl, halogen, or hydroxyl and
R6 is hydrogen or 1-4C-alkyl,
or a salt thereof.

6. A compound of the formula 1 as claimed in claim 1, in which
R1 is 1-4C-alkyl or 3-7C-cycloalkyl,
R2 is hydrogen, 1-4C-alkyl, halogen or hydroxy-1-4C-alkyl,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5- and R6-substituted phenyl radical,
where
R4 is hydrogen, 1-4C-alkyl, hydroxy-1-4C-alkyl, 1-4C-alkoxy, 1-4C-alkoxycarbonyl, carboxy-1-4C-alkyl, halogen, hydroxyl or trifluoromethyl,
R5 is hydrogen, 1-4C-alkyl, halogen, or hydroxyl and
R6 is hydrogen or 1-4C-alkyl,
or a salt thereof.

7. A compound of the formula 1 as claimed in claim 1, in which
R1 is 1-4C-alkyl,
R2 is hydrogen, 1-4C-alkyl, halogen or hydroxy-1-4C-alkyl,
R3 is hydroxy-1-4C-alkyl, 1-4C-alkoxy-1-4C-alkyl, carboxyl, 1-4C-alkoxycarbonyl or the radical —CO—NR31R32,
where
R31 is hydrogen, 1-4C-alkyl, hydroxy-1-4C-alkyl or 1-4C-alkoxy-1-4C-alkyl,
R32 is hydrogen or 1-4C-alkyl,
or where
R31 and R32 together and including the nitrogen atom to which they are attached form a pyrrolidino or morpholino radical,
Arom is phenyl,
or a salt thereof.

8. A compound of the formula 1 as claimed in claim 1, in which
R1 is 1-4C-alkyl,
R2 is hydrogen, 1-4C-alkyl, halogen or hydroxy-1-4C-alkyl,
R3 is carboxyl, 1-4C-alkoxycarbonyl or the radical —CO—NR31R32,
where
R31 is hydrogen, 1-4C-alkyl, hydroxy-1-4C-alkyl or 1-4C-alkoxy-1-4C-alkyl,
R32 is hydrogen or 1-4C-alkyl,
or where
R31 and R32 together and including the nitrogen atom to which they are attached form a pyrrolidino or morpholino radical,
Arom is phenyl,
or a salt thereof.

9. A compound of the formula 1 as claimed in claim 1, characterized by the formula 1-a, 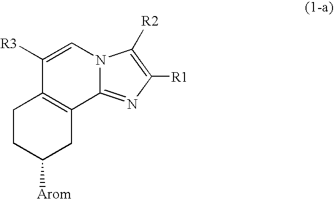
in which
R1 is hydrogen, 1-4C-alkyl, 3-7C-cycloalkyl, 1-4C-alkoxy-1-4C-alkyl, 1-4C-alkoxycarbonyl, 2-4C-alkenyl, 2-4C-alkinyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, hydroxy-3-4-C-alkenyl, hydroxy-3-4C-alkinyl, 1-4C-alkoxycarbonyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached are a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R4 is hydrogen, 1-4C-alkyl, hydroxy-1-4C-alkyl, 1-4C-alkoxy, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxyl,
R6 is hydrogen, 1-4C-alkyl or halogen and
R7 is hydrogen, 1-4C-alkyl or halogen,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
or a salt thereof.

10. A compound of the formula 1-a as claimed in claim 9,
in which
R1 is 1-4C-alkyl,
R2 is hydrogen, 1-4C-alkyl, halogen or hydroxy-1-4C-alkyl,
R3 is hydroxy-1-4C-alkyl, 1-4C-alkoxy-1-4C-alkyl, carboxyl, 1-4C-alkoxycarbonyl or the radical —CO—NR31R32,
where
R31 is hydrogen, 1-4C-alkyl, hydroxy-1-4C-alkyl or 1-4C-alkoxy-1-4C-alkyl,
R32 is hydrogen or 1-4C-alkyl,
or where
R31 and R32 together and including the nitrogen atom to which they are attached form a pyrrolidino or morpholino radical,
Arom is phenyl,
or a salt thereof.

11. A compound of the formula 1-a as claimed in claim 9,
in which
R1 is 1-4C-alkyl,
R2 is hydrogen, 1-4C-alkyl, halogen or hydroxy-1-4C-alkyl,
R3 is carboxyl, 1-4C-alkoxycarbonyl or the radical —CO—NR31R32,
where
R31 is hydrogen, 1-4C-alkyl, hydroxy-1-4C-alkyl or 1-4C-alkoxy-1-4C-alkyl,
R32 is hydrogen or 1-4C-alkyl,
or where
R31 and R32 together and including the nitrogen atom to which they are attached form a pyrrolidino or morpholino radical,
Arom is phenyl,
or a salt thereof.

12. A pharmaceutical composition comprising a compound as claimed in claim 1 and/or a pharmaceutically acceptable salt thereof together with a pharmaceutically acceptable auxiliary and/or excipient.

13. (canceled)

14. A method of preventing or treating a gastrointestinal disorder in a patient comprising administering a therapeutically effective amount of a compound of formula 1 according to claim 1 or a pharmaceutically acceptable salt thereof to a patient in need thereof.
